(19) United States
(12) Patent Application Publication
(10) Pub. No.: US 2003/0091602 A1
(43) Pub. Date: May 15, 2003

(54) METHOD OF TREATING A COSMETIC COMPOSITION BY IRRADIATION WITH NIR RADIATION, AND ITS USE

(76) Inventors: Helmut Witteler, Beindersheim (DE); Rainer Blum, Ludwigshafen (DE); Peter Hossel, Schifferstadt (DE); Axel Sanner, Frankenthal (DE); Reinhold Schwalm, Wachenheim (DE); Wilma M. Dausch, Limburgerhof (DE); Thomas Jaworek, Kallstadt (DE); Rainer Koniger, Freinsheim (DE)

Correspondence Address: KEIL & WEINKAUF, 1350 CONNECTICUT AVENUE, N.W., WASHINGTON, DC, 20036, US

(21) Appl. No.: 09/750,720

(22) Filed: Jan. 2, 2001

(30) Foreign Application Priority Data
Jan. 12, 2000 (DE) 10000807.0

(51) Int. Cl. A61K 7/00; C08J 3/28

(52) U.S. Cl. 424/401; 522/126; 522/130

(57) ABSTRACT

The present invention relates to a method of treating a cosmetic composition, which comprises treating the cosmetic composition with NIR radiation before, during or after application.

### DESCRIPTION

The present invention relates to a method of treating a cosmetic composition before, during or after application by irradiation with NIR radiation, and to the use of the resulting cosmetic composition for the treatment of skin, hair or nails. The cosmetic composition according to the invention is preferably used for setting, shaping and improving the structure of hair.

The present invention further relates to a cosmetic composition obtainable by NIR irradiation.

Methods for altering the properties of cosmetics during application by the action of heat, UV radiation or reactive chemicals are described widely in the prior art.

U.S. Pat. No. 3,820,550 describes the curing of N-substituted acrylamides, in particular of diacetone acrylamides, on the hair by heat, redox catalysts or autoxidation.

U.S. Pat. No. 3,472,604 describes the treatment of hair during coloring with water-soluble polymerizable vinyl monomers, the monomer being polymerized by means of an oxidizing agent.

U.S. Pat. No. 3,634,022 and U.S. Pat. No. 3,676,550 describe a method for the application of permanent waves, by, in addition to customary methods, olefinically unsaturated monomers or peroxide initiators are additionally used.

U.S. Pat. No. 4,278,659 describes hair treatment compositions comprising an acid, glyceraldehyde, resorcinol, and an oligomeric precondensate thereof, which are applied by means of heat.

U.S. Pat. No. 5,362,486 describes the polymerization of urethane acrylate oligomers on the hair, the polymerization reagents used being benzoyl peroxide and others.

U.S. Pat. No. 4,682,612 describes a process for the preparation of artificial nails which are cured by UVA radiation (320 to 400 nm)

A common factor of all of these processes is that they use methods which are unfavorable for contact with the human body because they can lead to skin irritations and to impairment of health. A further disadvantage of some of said processes is that the curing of the polymers or prepolymers used is not exactly controllable with regard to its point in time and its duration. In addition, it is a disadvantage of the treatment with UV light and heat radiation that this does not pass through the hair meaning that thorough and temporally optimized treatment of the cosmetic composition present on the hair is not possible.

The deliberate influencing of cosmetic preparations by the action of NIR radiation for their preparation or use is hitherto unknown. In particular, the use of cosmetic formulations together with NIR radiation such that, during use, polymers form or crosslink or the properties of polymers are changed, is unknown.

It is an object of the present invention to provide a method of treating cosmetic formulations which permits improved hair—and skincare and which does not have the disadvantages given above.

Surprisingly, we have now found that using near infrared (NIR) radiation it is possible to render effective virtually any system suitable for polymerization, curing or crosslinking, i.e. also those systems which are generally cured only with the help of UV radiation or long-wave infrared radiation. This is all the more surprising because the monomers, prepolymers and polymers given below much more strongly absorb UV radiation and long-wave infrared radiation than they do NIR radiation.

The particular advantage of the invention over the activation or polymerization of cosmetic compositions by heat, UV radiation, treatment with free radical formers, acids and bases is the simplified handling and the significantly reduced irritation of the parts of the body concerned. Compared with IR radiation, the advantage is that NIR radiation penetrates more deeply into the cosmetic composition than IR radiation and thus acts not only superficially. This is of advantage particularly when the cosmetic composition is to be activated, cured or polymerized only after application to or penetration into the substrate (e.g. skin or hair). Furthermore, in contrast to IR radiation, NIR radiation passes through the hair with significantly weaker absorption, meaning that NIR radiation is suitable precisely for voluminous hairstyles.

The invention firstly relates to a method of treating a cosmetic composition, which comprises treating the cosmetic composition before, during or after application with near infrared radiation (NIR radiation).

The invention further relates to a cosmetic composition obtainable by irradiation with NIR radiation, and to the use of the resulting composition for the treatment of skin, hair or nails.

The cosmetic compositions are, in particular, compositions having a film-forming, permeation-inhibiting, setting or shaping action, whose effect is activated, enhanced or accelerated during or after application by NIR radiation having an emission maximum in the wavelength range from 600 nm to 1500 nm, in particular 750 to 1100 nm.

The cosmetic composition is notable for the fact that, under the action of NIR radiation, at least one polymer is formed, where the polymer can be linear, branched or crosslinked; the formation of random and alternating copolymers, and of graft and block a 20 copolymers is also possible. The polymer is formed from any chemical compounds present in the cosmetic composition, preference being given to organic and organosilicon compounds. Particular preference is given to compounds which are capable of chemically reacting with polymerization, polyaddition, polycondensation or crosslinking or react with a combination of these reaction types. The reaction can also proceed with chemical modification of the treated structure (hair, nails, keratinous material, skin). In particular, the cosmetic composition comprises compounds from the following classes:

unsaturated compounds (in particular ethylenically unsaturated compounds),

epoxides

silicon compounds having at least one Si—O bond and optionally at least one Si—C bond

aromatic compounds which have at least one C—Obond with participation of a ring atom, for example phenol derivatives

formaldehyde and compounds which can cleave off formaldehyde

urotropine, melamine, urea and derivatives or structural derivatives thereof

substances which break down into free radicals under suitable conditions

in addition, the cosmetic composition can comprise isocyanates, alcohols, amines and their derivatives, carboxylic acids and their derivatives, lactams and lactones, carbodiimides.

All of the compounds from said classes can have low molecular weight or high molecular weight character. The only decisive factor is their ability to form, as a result of chemical reaction, substances of higher molecular weight or of higher mechanical strength or of lower permeability (for water or oxygen), or, as a result of chemical reaction with the treated structure, to bring about a corresponding modification of the properties of the treated structure. Polymers which crosslink under suitable conditions are likewise suitable as a constituent of the cosmetic formulations.

The cosmetic composition used according to the invention is preferably used for the treatment of skin, hair and nails, treatment of hair with a setting or fixing action being preferred.

Particularly suitable radiation sources for NIR radiation emit predominantly, but at least more strongly than commercially available infrared emitters, in the wavelength range from 600 nm to 1500 nm. NIR emitters can, for example, be halogen lamps, semiconductor lasers, light diodes, mercury vapor lamps or sodium vapor lamps. Commercially available NIR emitters are supplied, for example, by the companies IndustrieServis (Bruckmuhl) and Ushio (Tokyo).

In particular, the invention provides methods in which NIR radiation is used together with a formulation in hair cosmetics, preferably with preparations such as hair setting formulations, permanent wave preparations, hair treatments, hair lotions, hair rinses, hair emulsions, split-end fluids, neutralizers for permanent waves, hot-oil treatment preparations, conditioners, setting lotions, shampoos, hair colorants, agents for treating dandruff and hair loss, and in hair restorers. The method is preferably used for setting hair. The invention provides both for use in typical hair setting formulations, and also combination with other methods and agents for shaping hair, particularly using permanent waves.

In addition, the method according to the invention can also be used on the nails; in particular in conjunction with the use of nail varnishes.

The method according to the invention can also be used for cosmetic and dermatological compositions for the skin. Intended uses here are, in particular, skin preparations having a permeation-inhibiting action (permeation of water) and preparations for wound care.

Furthermore, the method according to the invention is used for cosmetic compositions for cleansing the skin. Such cosmetic cleansers are chosen from bar soaps, such as toilet soaps, curd soaps, transparent soaps, luxury soaps, deodorant soaps, cream soaps, baby soaps, skin protection soaps, abrasive soaps and syndets, liquid soaps, such as pasty soaps, soft soaps and washing pastes, and liquid washing, shower and bath preparations, such as washing lotions, shower preparations and gels, foam baths, oil baths and scrub preparations.

Furthermore, the method can be used for cosmetic compositions for the care and protection of the skin, in nailcare compositions, and in preparations for decorative cosmetics.

A further use of the method is to use in the case of skincare compositions, personal hygiene compositions, footcare compositions, light protection agents, repellents, shaving compositions, depilatories, antiacne compositions, make-up compositions, mascara, lipsticks, eye shadows, kohl pencils, eyeliners, blushers, powders and eyebrow pencils.

The skincare compositions are, in particular, in the form of W/O or O/W skin creams, day and night creams, eye creams, face creams, antiwrinkle creams, moisturizing creams, bleaching creams, vitamin creams, skin lotions, care lotions and moisturizing lotions.

Furthermore, the method is suitable for nose strips for pore cleansing, antiacne compositions, repellents, shaving compositions, depilatories, personal hygiene compositions, footcare compositions, and in babycare. The method is likewise suitable for the application of auxiliaries in pharmacy, preferably for the use of coatings and binders for solid dosage forms.

Depending on the field of application, the compositions according to the invention can be applied in a form suitable for skincare, such as, for example, as cream, foam, gel, gelspray, stick, powder, mousse, milk or lotion.

The method is preferably used in cosmetic compositions comprising the following classes of monomers, prepolymers and polymers:

Ethylenically unsaturated monomers which can be polymerized with a reaction initiated by free radicals are preferred. The term “ethylenically unsaturated” means that the monomers have at least: one polymerizable carbon-carbon double bond, which may be mono-, di-, tri-, or tetra-substituted.

The preferred ethylenically unsaturated monomers can be described by the following formula:

X—C (O) CR⁷=CHR⁶

where

X is chosen from the group of radicals —OH, —OM, —OR⁸, NH₂, —NHR⁸, N(R⁸)₂;

M is a cation chosen from the group consisting of: Na⁺, K⁺, Mg⁺⁺, Ca⁺⁺, Zn⁺⁺, NH₄+, alkylammonium, dialkylammonium, trialkylammonium and tetraalkylammonium;

the radicals R⁸ can be identical or different and are chosen from the group consisting of —H, C₁-C₄₀ linear- or branched-chain alkyl radicals, N,N-dimethylaminoethyl, 2-hydroxyethyl, 2-methoxyethyl, 2-ethoxyethyl, hydroxypropyl, methoxypropyl or ethoxypropyl.

R⁷ and R⁶ are, independently of one another, chosen from the group consisting of: —H, C₁-C₈ linear- or branched-chain alkyl chains, methoxy, ethoxy, 2-hydroxyethoxy, 2-methoxyethoxy and 2-ethoxyethyl.

Representative but nonlimiting examples of suitable monomers are, for example, acrylic acid and salts, esters and amides thereof. The salts can be derived from any desired non-toxic metal, ammonium or substituted ammonium counterions.

The esters can be derived from C₁-C₄₀ linear, C₃-C₄₀ branched-chain, or C₃-C₄₀ carbocyclic alcohols, from polyfunctional alcohols having 2 to about 8 hydroxyl groups, such as ethylene glycol, hexylene glycol, glycerol, and 1,2,6-hexanetriol, from aminoalcohols or from alcohol ethers, such as methoxyethanol and ethoxyethanol or polyethylene glycols.

Also suitable are N,N-dialkylaminoalkyl acrylates and methacrylates and N-dialkylaminoalkylacrylamide and -methacrylamide of the formula (II)

where R⁹=H, alkyl having 1 to 8 carbon atoms,

R¹⁰=H, methyl,

R¹¹=alkylene having 1 to 24 carbon atoms, optionally substituted by alkyl,

R¹², R¹³=C₁-C₄₀-alkyl radical,

Z=nitrogen when x=1, or oxygen when x=0.

The amides can be unsubstituted, N-alkyl or N-alkylamino monosubstituted, or N,N-dialkyl-substituted or N,N-dialkylamino disubstituted, where the alkyl or alkylamino groups are derived from C₁-C₄₀ linear, C₃-C₄₀ branched-chain, or C₃-C₄₀ carbocyclic units. Additionally, the alkylamino groups may be quaternized.

Preferred monomers of the formula II are N,N-dimethylaminomethyl (meth)acrylate, N,N-diethylaminomethyl (meth)acrylate, N,N-dimethylaminoethyl (meth)acrylate, N,N-diethylaminoethyl (meth) acrylate.

A monomer which can likewise be used is diacetone acrylamide (CAS number 2873-97-4).

Monomers which can likewise be used are substituted acrylic acids, and salts, esters and amides thereof, where the substituents on the carbon atoms are in the two or three position of the acrylic acid, and are chosen, independently of one another, from the group consisting of C₁-C₄-alkyl, —CN, COOH, particularly preferably methacrylic acid, ethacrylic acid and 3-cyanoacrylic acid. These salts, esters and amides of these substituted acrylic acids can be chosen as described above for the salts, esters and amides of acrylic acid.

Other suitable monomers are vinyl and allyl esters of C₁-C₄₀ linear, C₃-C₄₀ branched-chain or C₃-C₄₀ carbocyclic carboxylic acids (e.g.: vinyl acetate, vinyl propionate, vinyl neononanoate, vinyl neoundecanoic acid or vinyl t-butylbenzoate); vinyl or allyl halides, preferably vinyl chloride and allyl chloride, vinyl ethers, preferably methyl, ethyl, butyl, or dodecyl vinyl ether, vinylformamide, vinylmethylacetamide, vinylamine; vinyllactams, preferably vinylpyrrolidone and vinylcaprolactam, vinyl- or allyl-substituted heterocyclic compounds, preferably vinylpyridine, vinyloxazoline and allylpyridine.

Also suitable are N-vinylimidazoles of the formula III, in which R¹⁴ to R¹⁶, independently of one another, are hydrogen, C₁-C₄-alkyl or phenyl:

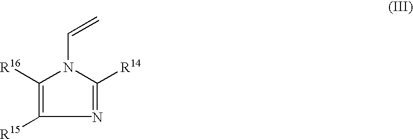

Further suitable monomers are diallylamines of the formula (IV)

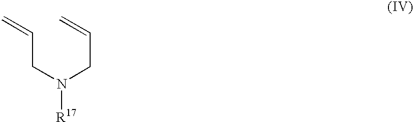

where R¹⁷ C₁-C₂₄-alkyl.

Other suitable monomers are vinylidene chloride; and hydrocarbons having at least one carbon-carbon double bond, preferably styrene, alpha-methylstyrene, tert-butylstyrene, butadiene, isoprene, cyclohexadiene, ethylene, propylene, 1-butene, 2-butene, isobutylene, vinyltoluene, and mixtures of these monomers.

Particularly suitable monomers are acrylic acid, methacrylic 35 acid, ethylacrylic acid, methyl acrylate, ethyl acrylate, propyl acrylate, n-butyl acrylate, isobutyl acrylate, t-butyl acrylate, 2-ethylhexyl acrylate, decyl acrylate, methyl methacrylate, ethyl methacrylate, propyl methacrylate, n-butyl methacrylate, iso-butyl methacrylate, t-butyl methacrylate, 2-ethylhexyl methacrylate, decyl methacrylate, methyl ethacrylate, ethyl ethacrylate, n-butyl ethacrylate, isobutyl ethacrylate, t-butyl ethacrylate, 2-ethylhexyl ethacrylate, decyl ethacrylate, 2,3-dihydroxypropyl acrylate, 2,3-dihydroxypropyl methacrylate, 2-hydroxyethyl acrylate, hydroxypropyl acrylates, 2-hydroxyethyl methacrylate, 2-hydroxyethyl ethacrylate, 2-methoxyethyl acrylate, 2-methoxyethyl methacrylate, 2-methoxyethyl ethacrylate, 2-ethoxyethyl methacrylate, 2-ethoxyethyl ethacrylate, hydroxypropyl methacrylates, glyceryl monoacrylate, glyceryl monomethacrylate, polyalkylene glycol (meth)acrylates, unsaturated sulfonic acids, such as, for example, acrylamidopropanesulfonic acid; acrylamide, methacrylamide, ethacrylamide, N-methylacrylamide,

N,N-dimethylacrylamide, N-ethylacrylamide, N-isopropylacrylamide, N-butylacrylamide, N-t-butylacrylamide, N-octylacrylamide, N-t-octylacrylamide, N-octadecylacrylamide, N-phenylacrylamide, N-methylmethacrylamide, N-ethylmethacrylamide, N-dodecylmethacrylamide, 1-vinylimidazole, 1-vinyl-2-methylimidazole, N,N-dimethylaminomethyl (meth)acrylate, N,N-diethylaminomethyl (meth)acrylate, N,N-dimethylaminoethyl (meth)acrylate, N,N-diethylaminoethyl (meth)acrylate, N,N-dimethylaminobutyl (meth)acrylate, N,N-diethylaminobutyl (meth)acrylate, N,N-dimethylaminohexyl (meth)acrylate, N,N-dimethylaminooctyl (meth)acrylate, N,N-dimethylaminododecyl (meth)acrylate, N-[3-(dimethylamino)propyl]methacrylamide, N-[3-(dimethylamino)propyl]acrylamide, N-[3-(dimethylamino)pbutyl]methacrylamide, N-[8-(dimethylamino)octyl]methacrylamide, N-[12-(dimethylamino)dodecyl]methacrylamide, N-[3-(diethylamino)propyl]methacrylamide, N-[3-(diethylamino)propyl]acrylamide;

maleic acid, fumaric acid, maleic anhydride and its half-esters, crotonic acid, itaconic acid, diallyldimethylammonium chloride, vinyl ethers (for example: methyl, ethyl, butyl or dodecyl vinyl ether), vinylformamide, vinylmethylacetamide, vinylamine; methyl vinyl ketone, maleimide, vinylpyridine, vinylimidazole, vinylfuran, styrene, styrene sulfonate, allyl alcohol, and mixtures thereof.

Of these, particular preference is given to acrylic acid, methacrylic acid, maleic acid, fumaric acid, crotonic acid, maleic anhydride, and its half-esters, methyl acrylate, methyl methacrylate, ethyl acrylate, ethyl methacrylate, n-butyl acrylate, n-butyl methacrylate, t-butyl acrylate, t-butyl methacrylate, isobutyl acrylate, isobutyl methacrylate, 2-ethylhexyl acrylate, N-t-butylacrylamide, N-octylacrylamide, 2-hydroxyethyl acrylate, hydroxypropyl acrylate, 2-hydroxyethyl methacrylate, hydroxypropyl methacrylate, alkylene glycol (meth)acrylates, unsaturated sulfonic acids, such as, for example, acrylamidopropanesulfonic acid, vinylpyrrolidone, vinylcaprolactam, vinyl ethers (e.g.: methyl, ethyl, butyl or dodecyl vinyl ethers), vinylformamide, vinylmethylacetamide, vinylamine, 1-vinylimidazole, 1-vinyl-2-methylimidazole, N,N-dimethylaminoinethyl methacrylate and N-[3-(dimethylamino)propyl]methacrylamide; 3-methyl-l-vinylimidazolium chloride, 3-methyl-1-vinylimidazolium methylsulfate, N,N-dimethylaminoethyl methacrylate, N-[3-(dimethylamino)propyl]methacrylamide quaternized with methyl chloride, methyl sulfate or diethyl sulfate.

Monomers with a basic nitrogen atom can be quaternized in the following way:

Substances suitable for the quaternization of the amines are, for example, alkyl halides having 1 to 24 carbon atoms in the alkyl group, e.g. methyl chloride, methyl bromide, methyl iodide, ethyl chloride, ethyl bromide, propyl chloride, hexyl chloride, dodecyl chloride, lauryl chloride and benzyl halides, in particular benzyl chloride and benzyl bromide. Further suitable quaternizing agents are dialkyl sulfates, in particular dimethyl sulfate or diethyl sulfate. The quaternization of the basic amines can also be carried out with alkylene oxides, such as ethylene oxide or propylene oxide, in the presence of acids. Preferred quaternizing agents are: methyl chloride, dimethyl sulfate or diethyl sulfate.

The quaternization can be carried out before the polymerization or after the polymerization.

Furthermore, the reaction products of unsaturated acids, such as, for example, acrylic acid or methacrylic acid, with a quaternized epichlorohydrin of the formula (V) (R¹⁸=C₁-C₄₀-alkyl) can be used.

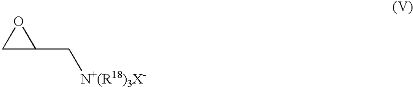

Examples thereof are:

(meth)acryloyloxyhydroxypropyltrimethylammonium chloride and (meth)acryloyloxyhydroxypropyltriethylammonium chloride.

The monomers used can, provided they contain ionizable groups, be partially or completely neutralized before or after the polymerization with acids or bases in order, for example, to adjust the water solubility or dispersibility to a desired degree.

Neutralizing agents which can be used for monomers, prepolymers or polymers carrying acid groups are, for example, mineral bases, such as sodium carbonate, alkali metal hydroxides, and ammonia, organic bases, such as aminoalcohols, specifically 2-amino-2-methyl-1-propanol, monoethanolamine, diethanolamine, triethanolamine, triisopropanolamine, tri[(2-hydroxy)-1-propyl]amine, 2-amino-2-methyl-1,3-propanediol, 2-amino-2-hydroxymethyl-1,3-propanediol, and diamines such as, for example, lysine.

Neutralizing agents which can be used for monomers, prepolymers or polymers carrying cationizable groups are, for example, mineral acids, such as hydrochloric acid, sulfuric acid or phosphoric acid, and organic acids, such as carboxylic acids, lactic acid, citric acid or others.

In addition to the abovementioned monomers, the monomers used may also be macromonomers such as, for example, silicone-containing macromonomers having one or more free-radically polymerizable groups. Macromonomers of this type are described, for example, in EP 0408311 (A2, B1) and EP 0412704 (A2, B1), to which reference is expressly made here.

Furthermore, it is also possible to use fluorine-containing monomers, as are described, for example, in EP 558423 (A1, B1), crosslinking compounds or compounds which regulate the molecular weight, in combination or alone.

Regulators which can be used are the customary compounds known to the person skilled in the art, for example, sulfur compounds (e.g.: mercaptoethanol, 2-ethylhexyl thioglycolate, thioglycolic acid or dodecyl mercaptan), and tribromochloromethane or other compounds which have a regulating action on the molecular weight of the resulting polymers.

Where appropriate, it is also possible to use silicone compounds which contain thiol groups.

The crosslinking monomers which can be used are compounds with at least two ethylenically unsaturated double bonds, such as, for example, esters of ethylenically unsaturated carboxylic acids, such as acrylic acid or methacrylic acid and polyhydric alcohols, ethers of at least dihydric alcohols, such as, for example, vinyl ether or allyl ether. Also suitable are straight-chain or branched, linear or cyclic aliphatic or aromatic hydrocarbons which have at least two double bonds which, in the case of the aliphatic hydrocarbons, must not be conjugated. Also suitable are amides of acrylic and methacrylic acid and N-allylamines of at least difunctional amines, such as, for example, 1,2-diaminoethane, 1,3-diaminopropane. Also suitable are triallylamine or corresponding ammonium salts, N-vinyl compounds of urea derivatives, at least difunctional amides, cyanurates or urethanes. Other suitable crosslinkers are divinyldioxane, tetraallylsilane or tetravinylsilane.

Particularly preferred crosslinkers are, for example, methylenebisacrylamide, triallylamine and triallylammonium salts, divinylimidazole, N,N′-divinylethyleneurea, reaction products of polyhydric alcohols with acrylic acid or methacrylic acid, methacrylic esters and acrylic esters of polyalkylene oxides or polyhydric alcohols which have been reacted with ethylene oxide and/or propylene oxide and/or epichlorohydrin.

Further cosmetic formulations which are used advantageously for the method comprise ethylenically unsaturated prepolymers and additionally, where appropriate, one or more reactive thinners.

The prepolymers used are, for example, (meth)acrylate-functional (meth)acrylic copolymers, polyether (meth)acrylates, polyester (meth)acrylates, unsaturated polyesters, epoxy (meth)acrylates, urethane (meth)acrylates, amino (meth)acrylates, melamine (meth)acrylates, silicone (meth)acrylates. Preference is given to using urethane (meth)acrylates, polyester (meth)acrylates and/or aliphatic urethane acrylates. The prepolymers usually have a number-average molecular weight of from 500 to 50000, preferably from 500 to 5000.

Preference is given to using prepolymers which have at least 2, particularly preferably 3 to 6, double bonds. Furthermore, the binders used preferably have a double bond equivalent weight of from 400 to 2000, particularly preferably from 500 to 900.

Furthermore, the binders preferably have a viscosity of from 250 to 11000 mPas.

Polyester (meth)acrylates are in principle known to the person skilled in the art. They can be prepared by a variety of methods. For example, acrylic acid and/or methacrylic acid can be used directly as acid component for building up the polyester. It is also possible to use hydroxyalkyl esters of (meth)acrylic acid as alcohol component directly for building up the polyester. However, preference is given to preparing the polyester (meth)acrylates by acrylating polyesters. For example, hydroxyl-containing polyesters can firstly be built up, which are then reacted with acrylic or methacrylic acid. It is also possible to firstly build up carboxyl-containing polyesters, which are then reacted with a hydroxyalkyl ester of acrylic or methacrylic acid. Unreacted (meth)acrylic acid can be removed from the reaction mixture by washing out, distillation or, preferably, by reaction with an equivalent amount of a mono- or diepoxide compound using suitable catalysts, such as, for example, triphenylphosphine.

Polyether (meth)acrylates are likewise in principle known to the person skilled in the art. They can be prepared by a variety of methods. For example, hydroxyl-containing polyethers, which are esterified with acrylic acid and/or methacrylic acid, can be obtained by reacting di- and/or polyhydric alcohols with varying amounts of ethylene oxide and/or propylene oxide by well known methods (cf. e.g. Houben-Weyl, Volume XIV, 2, Makromolekulare Stoffe II [Macromolecular Substances II] (1963)). It is also possible to use polymerization products of tetrahydrofuran or butylene oxide.

A flexibilization of the polyether (meth)acrylates and of the polyester (meth)acrylates is possible, for example, by reacting corresponding OH-functional prepolymers or oligomers (polyether-or polyester-based) with longer-chain, aliphatic dicarboxylic acids, in particular aliphatic dicarboxylic acids having at least 6 carbon atoms, such as, for example, adipic acid, sebacic acid, dodecanedioic acid and/or dimer fatty acids. This flexibilization reaction can be carried out here before or else after the addition of acrylic or methacrylic acid to the oligomers or prepolymers.

Furthermore, epoxy (meth)acrylates are also well known to the person skilled in the art as coatings and therefore do not need to be explained in more detail. They are usually prepared by the addition of acrylic acid to epoxy resins, for example epoxy resins based on bisphenol A or other commercially available epoxy resins.

A flexibilization of the epoxy (meth)acrylates is, for example, analogously possible by reacting corresponding epoxy-functional prepolymers or oligomers with longer-chain, aliphatic dicarboxylic acids, in particular aliphatic dicarboxylic acids having at least 6 carbon atoms, such as, for example, adipic acid, sebacic acid, dodecanedioic acid and/or dimer fatty acids. This flexibilization reaction can be carried out here before or after the addition of acrylic or methacrylic acid to the oligomers or prepolymers. Urethane (meth)acrylates are likewise well known to the person skilled in the art and therefore do not need to be explained in further detail. They can be obtained by reacting a di- or polyisocyanate with a chain-extending agent from the group of diols/polyols and/or diamines/polyamines and/or dithiols/polythiols and/or alkanolamines, and subsequently reacting the remaining free isocyanate groups with at least one hydroxyalkyl (meth)acrylate or hydroxyalkyl ester of other ethylenically unsaturated carboxylic acids.

The amounts of chain-extending agents, di- or polyisocyanate and hydroxyalkyl ester are preferably chosen here such that

1. the equivalent ratio of NCO groups to the reactive groups of the chain-extending agent (hydroxyl, amino or mercaptyl groups) is between 3:1 and 1:2, preferably 2:1, and

2. the OH groups of the hydroxyalkyl esters of the ethylenically unsaturated carboxylic acids are present in stoichiometric amounts relative to the isocyanate groups of the prepolymer from isocyanate and chain-extending agent which are still free.

Furthermore, it is possible to prepare the polyurethane acrylates by firstly reacting some of the isocyanate groups of a di- or polyisocyanate with at least one hydroxyalkyl ester, and then reacting the remaining isocyanate groups with a chain-extending agent. In this case too, the amounts of chain-extending agent, isocyanate and hydroxyalkyl ester are chosen such that the equivalent ratio of the NCO groups to the reactive groups of the chain-extending agent is between 3:1 and 1:2, preferably 2:1, and of the hydroxyalkyl ester is 1 : 1. All intermediate forms of these two processes are of course also possible. For example, some of the isocyanate groups of a diisocyanate can firstly be reacted with a diol, then some more of the isocyanates can be reacted with the hydroxyalkyl ester and, after this, the remaining isocyanate groups can be reacted with a diamine.

These different preparation processes for polyurethane acrylates are known (cf. e.g. EP-A-203 161) and therefore do not require more detailed description.

A flexibilization of the urethane (meth)acrylates is, for 45 example, possible by reacting corresponding isocyanate-functional prepolymers with longer-chain, aliphatic diols and/or diamines, in particular aliphatic diols and/or diamines having at least 6 carbon atoms. This flexibilizatlon reaction can be carried out here before or after the addition of acrylic or methacrylic acid to the oligomers or prepolymers.

The prepolymers can, where appropriate, be used together with one or more reactive thinners. The reactive thinners can here be ethylenically unsaturated compounds. The reactive thinners can be mono-, di- or polyunsaturated. They are usually used to influence the rheological behavior and the material properties.

The reactive thinner(s) is/are preferably used in the prepolymers in an amount of from 0 to 70% by weight, particularly preferably from 15 to 65% by weight, in each case based on the total weight of the prepolymer.

Examples of the reactive thinners used are (meth)acrylic acid and esters thereof, maleic acid and esters or half-esters thereof, vinyl acetate, vinyl ethers, vinylureas and the like. Examples are alkylene glycol di(meth)acrylate, polyethylene glycol di(meth)acrylate, 1,3-butanediol di(meth)acrylate, vinyl (meth)acrylate, allyl (meth)acrylate, glycerol tri(meth)acrylate, trimethylolpropane tri(meth)acrylate, trimethylolpropane di(meth)acrylate, styrene, vinyltoluene, divinylbenzene, pentaerythritol tri(meth)acrylate, pentaerythritol tetra(meth)acrylate, dipropylene glycol di(meth)acrylate, hexanediol di(meth)acrylate, ethoxyethoxyethyl acrylate, N-vinylpyrrolidone, phenoxyethyl acrylate, dimethylaminoethyl acrylate, hydroxyethyl (meth)acrylate, butoxyethyl acrylate, isobornyl (meth)acrylate, dimethylacrylamide and dicyclopentyl acrylate, the long-chain linear diacrylates having a molecular weight from 400 to 4000, preferably from 600 to 2500, described in EP-A-250 631. For example, the two acrylate groups can be separated by a polyoxybutylene structure. It is also possible to use 1,12-dodecyl diacrylate and the reaction product of 2 mols of acrylic acid with one mol of a dimer fatty alcohol, which generally has 36 carbon atoms. Also suitable are mixtures of said monomers.

The reactive thinners used are preferably mono- and/or 40 diacrylates, such as, for example, isobornyl acrylate, hexanediol diacrylate, tripropylene glycol diacrylate and Laromere™ 8887 from BASF Aktiengesellschaft. Particular preference is given to isobornyl acrylate, hexanediol diacrylate and tripropylene glycol diacrylate.

Other cosmetic formulations which can be used advantageously for the method comprise unsaturated compounds derived from unsaturated fatty acids; for example triglycerides and alkyd resins (cf. DIN 53183). The alkyd resins can be formulated, for example, as alkyd varnishes (cf. DIN 55945).

Furthermore, the coatings according to the invention optionally also comprise customary auxiliaries and/or additives, for example light protection agents (e.g. HALS compounds, benzotriazoles, oxalanilide and the like), slip additives, polymerization inhibitors, matting agents, antifoams, flow-control agents and film-forming auxiliaries, e.g. cellulose derivatives, or other additives.

The invention also relates to the use of cosmetic formulations which comprise phenolic, melamine and formaldehyde resins, or comprise the substances from which these resins form under the action of NIR radiation. Particularly suitable are crosslinking systems as given in U.S. 4,588,760 and U.S. 4,278,659 and to which reference is expressly made here. Other particularly suitable ingredients are the polymers and dispersions described in DE-A 198 165 27.

The invention further relates to the use of cosmetic formulations which, under the action of NIR radiation, form macromolecules having Si—O—Si groups. Particularly suitable for this purpose are the ingredients and formulations given in DE-A 198 227 22, to which reference is expressly made here.

The cosmetic formulations according to the invention can also comprise dyes. Particularly suitable dyes are those which absorb NIR radiation. NIR-absorbing dyes are known to the person skilled in the art and described in the literature. Particular preference is given here to NIR polymerization initiators and NIR photocatalysts as described in EP-A 774 492, U.S. Pat. No. 5,607,814, U.S. Pat. No. 5,686,639, U.S. Pat. No. 5,858,604 and the literature cited therein, and to which reference is expressly made here.

For example, cationic dyes which contain at least one of the following groups

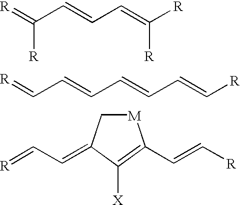

where

R stands for identical or different aromatic or quinoid radicals,

X is —Cl or —N(phenyl)₂ and

M is —CH₂- or —CH₂—CH₂—are suitable An example of such compounds is the cationic NIR dye (1):

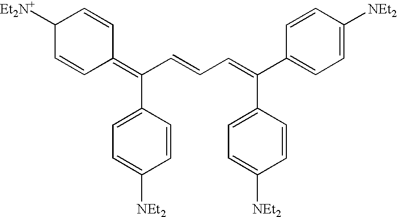

To improve the effectiveness of the NIR radiation, compounds which contain R—O—O—R or R′—N═N—R′ groups can be added to the abovementioned monomers, prepolymers and polymers. This is of interest particularly when the compounds contain ethylenically unsaturated groups. R stands for identical or different organic radicals or for hydrogen. R′ stands for identical or different organic radicals. Preference is given to hydrogen peroxide, hydroperoxides, peroxides, percarbonates and peresters. In addition, inorganic percompounds, such as sodium, potassium and ammonium peroxodisulfate, can be added. Further suitable initiators are the redox initiator systems known to the person skilled in the art, e.g. Fe(II)/H₂O₂, tartaric acid/H₂O₂, t-butyl hydroperoxide/tartaric acid, ascorbic acid/H₂O₂ or t-butyl hydroperoxide/ascorbic acid.

Suitable compounds of the R—O—O—R type are, for example, acetylcyclohexanesulfonyl peroxide, dicetyl peroxydicarbonate, diisopropyl peroxydicarbonate, t-amyl perneodecanoate, t-butyl perneodecanoate, bis(2,4-dichlorobenzoyl) peroxide, t-butyl perpivalate, bis(3,5,5-trimethylhexanoyl) peroxide, dioctanoyl peroxide, didecanoyl peroxide, dilauroyl peroxide, bis(2-methylbenzoyl) peroxide, succinyl peroxide, diacetyl peroxide, dibenzoyl peroxide, t-butyl per-2-ethylhexanoate, bis(4-chlorobenzoyl) peroxide, t-butyl perisobutyrate, t-butyl permaleate, 1,1-bis(t-butylperoxy)-3,5,5-trimethylcyclohexane, 1,1-bis(t-butylperoxy)cyclohexane, t-butyl peroxyisopropylcarbonate, t-butyl per-3,5,5-trimethylhexanoate, 2,5-dimethylhexane 2,5-diperbenzoate, t-butyl peracetate, t-butyl perbenzoate, 2,2-bis(t-butylperoxy)butane, 2,2-bis(t-butylperoxy)propane, dicumyl peroxide, 2,5-dimethylhexane-2,5-di-t-butyl peroxide, 3-t-butylperoxy-3-phenylphthalide, di-t-amyl peroxide, α,α′-bis(t-butylperoxyisopropyl)benzene, 3,5-bis(t-butylperoxy)-3,5-dimethyl-1,2-dioxolane, di-t-butyl peroxide, 2,5-dimethyl-3-hexyne-2,5-di-t-butyl peroxide, 3,3,6,6,9,9-hexamethyl-1,2,4,5-tetraoxacyclononane, p-methane hydroperoxide, pinane hydroperoxide, diisopropylbenzene mono-α-hydroperoxide, cumene hydroperoxide, t-butyl hydroperoxide, dimyristyl peroxydicarbonate, dicyclohexyl peroxydicarbonate, bis(4-t-butylcyclohexyl) peroxydicarbonate, di-n-butyl peroxydicarbonate, di-2-ethylhexyl peroxydicarbonate, diisotridecyl peroxydicarbonate.

Suitable compounds of the R′—N═N—R′ type are, for example, 2,2′-azobis(4-methoxy-2,4-dimethylvaleronitrile), 2,2′-azobis(2,4-dimethylvaleronitrile), 2,2′-azobis(isobutyronitrile), dimethyl 2,2′-azobis(isobutyrate), 2,2′-azobis(2-methylbutyronitrile), 1,1′-azobis(l-cyclohexanecarbonitrile), 2-(carbamoylazo)-isobutyronitrile, 2,2′-azobis(2,4,4-trimethylpentane), 2,2′-azobis(N,N′-dimethyleneisobutyramidine) dihydrochloride, 2,2′-azobis(2-amidinopropane) dihydrochloride, 2,2′-azobis-(N,N′-dimethyleneisobutyramidine), 4,4′-azobis(4-cyanopentanoic acid), 2,2′-azobis[2-methyl-N-(2-hydroxyethyl)propionamide].

Instead of compounds of the R—O—O—R and R′—N═N—R′ type, compounds R—R which decompose into free radicals under conditions typical for use are also suitable, for example 3,4-dimethyl-3,4-diphenylhexane and 3,4-dimethyl-3,4-diphenylbutane.

To improve the effectiveness of the NIR radiation, the siccatives known to the person skilled in the art can be added to the abovementioned monomers, prepolymers and polymers. These are drying agents, in particular for unsaturated compounds and their derivatives (e.g. alkyd resins, triglycerides). An example thereof is manganese(II) acetate. Siccatives whose action is intensified by NIR radiation are particularly suitable.

The following additives are used in the cosmetic compositions for the method according to the invention particularly when the abovementioned monomers, prepolymers and polymers contain epoxide groups:

Aliphatic polyamines H₂N(CH₂CH₂NH)ₙH, e.g diethylenetriamines (DETA, n=2) and triethylenetetramines (TETA, n=3), aliphatic diamines based on propylene oxide and ammonia H₂N(CH₂CH(CH₃)O)ₙCH₂CH(CH₃)NH_{2,} e.g. Jeffamine D-230 (Texaco Chemical Co.) where n=2 to 3, polyamidediamines or amidopolyamines (e.g. from the reaction of dimer fatty acids with DETA or TETA), cycloaliphatic diamines, e.g. cyclohexane-1,2-diamine, 4-(2-aminopropan-2-yl)-1-methylcyclohexan-1-amine, or menthanediamine, aromatic diamines, e.g. bis(4-aminophenyl)methane (MDA or methylene dianiline) and bis(4-aminophenyl) sulfones (DADS, DDS, or dapsone), carboxylic anhydrides, e.g. methylbicyclo[2.2.1]-heptene-2,3-dicarboxylic anhydride, 1,2-cyclohexanedicarboxylic anhydride, hexahydrophthalic anhydride or phthalic anhydride, tertiary amines NR₃, where R₃ are identical or different organic radicals (preferably alkyl radicals), melamine, urea and phenolformaldehyde adducts (also aminoplasts, phenoplasts), polycarboxylic polyesters, dicyandiamide, Lewis acids and bases, e.g. boron trifluoride complexed with methylethylamine, 2,4,6-tris(N,N-dimethylaminomethyl)phenol (Rohm & Haas DMP-30), N,N-dimethylbenzylamines.

The abovementioned polymers, prepolymers and monomers are present in the cosmetic composition to be used according to the invention in an amount of from 0.05 to 99% by weight, preferably 0.1 to 50% by weight and particularly preferably in an amount of from 0.2 to 20% by weight. Ina particularly preferred embodiment, the cosmetic compositions additionally comprise more than 50% by weight of fluids which can, for example, act as solvents or propellants. Examples of these fluids are water, ethanol, isopropanol, propane, butane, dimethyl ether, diethyl ether, carbon dioxide, compressed air, hexafluoroethane and decamethylcyclopentasiloxane.

The cosmetic formulations used according to the invention can comprise, as well as the abovementioned substances and their mixtures, auxiliaries which are customary in cosmetics, such as emollients, film-forming auxiliaries, pigments, perfumes, thickeners, surfactants, preservatives, cosmetic active ingredients, such as phytantriol, vitamins and provitamins, for example vitamin A, E and C, retinol, bisabolol, panthenol, natural and synthetic light protection agents, natural substances, propellants, solubilizers, repellents, bleaches, colorants, tinting agents, tanning agents, reflectors, proteins, ceramide, AHAs (alpha-hydroxycarboxylic acids, such as, for example, lactic acid and salicylic acid), and salts thereof, fruit acids, collagen, protein hydrolysates, stabilizers, pH regulators, emulsifiers, gel formers, bodying agents, silicones, moisturizers, refatting agents, UV protectants or other customary additives, alone or in combination.

The auxiliaries can be present during the polymerization and/or added after the polymerization.

Other customary additives which may be present are fatty substances, such as mineral and synthetic oils, such as, for example, paraffins, silicone oils and aliphatic hydrocarbons having more than 8 carbon atoms, animal and vegetable oils, such as, for example, sunflower oil, coconut oil, avocado oil, olive oil, lanolin, or waxes, fatty acids, fatty acid esters, such as, for example, triglycerides of C₆—C₃₀-fatty acids, wax esters, such as, for example, jojoba oil, fatty alcohols, vaseline, hydrogenated lanolin. It is of course also possible to use mixtures thereof.

Customary thickeners in such formulations are crosslinked polyacrylic acids and derivatives thereof, polysaccharides, such as xanthan gum, agar agar, alginates or Tyloses, cellulose derivatives, e.g. carboxymethylcellulose or hydroxycarboxymethylcellulose, fatty alcohols, monoglycerides and fatty acids, polyvinyl alcohol and polyvinylpyrrolidone.

It is also possible to use antimicrobial agents. These generally include all suitable preservatives having specific action against Gram-positive bacteria, e.g. triclosan (2,4,4′-trichloro-2′-hydroxydiphenyl ether), chlorhexidin (1,1′-hexamethylenebis[5-(4-chlorophenyl)biguanide), and TTC (3,4,41-trichlorocarbanilide). Quaternary ammonium compounds are in principle likewise suitable, but are preferably used for disinfecting soaps and washing lotions. Numerous fragrances also have antimicrobial properties. Special combinations having particular activity against Gram-positive bacteria are used for the composition so-called deoperfumes. A large number of essential oils or characteristic ingredients thereof, such as, for example, oil of cloves (eugenol), mint oil (menthol) or thyme oil (thymol), also exhibit excellent antimicrobial effectiveness. The antibacterial substances are preferably used in concentrations of from about 0.1 to 0.3% by weight.

Suitable solvents which are to be mentioned in particular are water and lower monoalcohols or polyols having 1 to 6 carbon atoms and mixtures thereof; preferred monoalcohols or polyols are ethanol, i-propanol, propylene glycol, glycerol and sorbitol. Further preferred auxiliaries are solvents and propellants from the group consisting of dimethyl ether, propane, butane, fluorinated hydrocarbons, cyclic silicones, branched and unbranched alkanes, organic esters and ethers.

The cosmetic formulations used according to the invention can, as well as the abovementioned substances and their mixtures, also comprise additional polymers, such as, for example, polyamides, polyurethanes, polyesters, homo- and copolymers of ethylenically unsaturated monomers. Preferred homo- and copolymers are obtainable from the abovementioned monomers. These can be functionalized by carboxylate groups, sulfonate groups, phosphonate groups, and by nitrogen-containing cationic groups.

In addition, silicones and alkoxylated silicones and compounds derived therefrom may be present. Examples of such polymers, some of which are also used in cosmetics, are listed below:

| INCI/CTFA name | Polymer | Manufacturer |
|---|---|---|
| Acrylate Copolymer | Amerhold | Amerchol |
| PVP/Acrylate Copolymer | Luviflex VBM 35 | BASF |
| PVP/VA | Luviskol VA | BASF |
| Polyvinylcaprolactam | Luviskol Plus | BASF |
| VA/Crotonate Copolymer | Luviset CA 66 | BASF |
| VA/Crotonate/Vinyl propionate Copolymer | Luviset CAP | BASF |
| Acrylate/Acrylamide Copolymer | Ultrahold 8 | BASF |
| Acrylate/Acrylamide Copolymer | Ultrahold Strong | BASF |
| Acrylate Copolymer | Luvimer MAE | BASF |
| Acrylate Copolymer | Luvimer 100P, 36D, 30E | BASF |
| Polyquaternium 46 | Luviquat Hold | BASF |
| Polyurethane-1 | Luviset P.U.R. | BASF |
| Methacryloyl ethylbetaine/Acrylate | Diaformer | Clariant |
| Copolymer | | |
| Diglycol/CHDM/Isophthalate/SIP | Eastman AQ Polymer | Eastman |
| Copolymer | | |
| Acrylate/Diacetone acrylamide Copolymer | Plascise L53 | Goo Chemicals |
| Butyl acrylate-Lauryl Methacrylate | Diahold EX-55/Plascise L | Goo Chemicals |
| | 1210 | |
| PVP | PVP K | ISP |
| PVP/VA | PVP/PA | ISP |
| Vinylcaprolactam/PVP/Dimethylaminomethyl | Copolymer VC 713 | ISP |
| Methacrylate Copolymer | (= Advantage HC) | |
| Vinylcaprolactam/PVP/Dimethylaminomethyl | H2OLD EP-1 | ISP |
| Methacrylate Copolymer | | |
| PVM/MA Butyl ester Copolymer | Gantrez ES 425 | ISP |
| VA/Butyl maleate/Isobornyl Acrylate | Advantage Plus (CP, V) | ISP |
| Copolymer | | |
| PVM/MA Ethyl ester Copolymer | Omnirez 2000 | ISP |
| PVP/DMAPA Acrylate Copolymer | Styleeze CC-10 | ISP |
| PVP/Vinylcaprolactam/DMAPA Acrylate | Aquaflex SF-40 | ISP |
| Copolymer | | |
| N-Methylacryloyl oxethyl | Yukaformer R205 | Mitsubishi |
| N,N-dimethylammonium-a-N-methyl- | | |
| carboxybetaine alkyl methacrylate | | |
| N-Methylacryloyl oxethyl | Yukaformer SM | Mitsubishi |
| N,N-dimethylammonium-a-N-methyl- | | |
| carboxybetaine alkyl methacrylate | | |
| VA/Crotonate/Copolymer | Resyn 28-1310 | National Starch |
| VA/Crotonate/Neodecanoate Copolymer | Resyn 28-2930 | National Starch |
| Octylacrylamide/Acrylate/ | Amphomer 28-4910 | National Starch |
| Butylaminoethyl Methacrylate Copolymer | | |
| Octylacrylamide/Acrylate/ | Amphomer LV-71 | National Starch |
| Butylaminoethyl Methacrylate Copolymer | | |
| Acrylate/Octylacrylamide Copolymer | Amphomer HC 28-4942 | National Starch |
| | (= Versatyl) | |
| Octylacrylamide/Acrylate Copolymer | Versatyl 90 | National Starch |
| Acrylate Copolymer | Balance 0/55 | National Starch |
| Octylacrylamide/Acrylate/Butylaminoethyl | Balance 47 (= Lovocryl 47) | National Starch |
| Methacrylate Copolymer | | |
| Acrylate/Hydroxy ester Acrylate Copolymer | Acudyne | Rohm & Haas |
| Diglycol/CHDM/Isophthalates/SIP | Eastman AQ | Eastman |
| Copolymer | | |
| Polyurethane-1 | Luviset P.U.R | BASF |
| PEG/PPG-25/25 Dimethicone/Acrylates | Luviflex Silk | BASF |
| Copolymer | | |

The abovementioned polyamides are described in EP 0696607 (A1) and in EP 0787480 (A1).

Further suitable cationic polymers: Those with the INCI name Polyquaternium, e.g. copolymers of vinylpyrrolidone/N-vinylimidazolium salts (Luviquat™ FC, Luviquat™ HM, Luviquate™ MS, Luviquat™ Care), copolymers of N-vinylpyrrolidone/dimethylaminoethyl methacrylate, quaternized with diethyl sulfate (Luviquate™ PQ 11), copolymers of N-vinylcaprolactam/N-vinylpyrrolidone/N-vinylimidazolium salts (Luviquat™ Hold); cationic cellulose derivatives (polyquaternium-4 and 10), acrylamide copolymers (polyquaternium-[lacuna]), Styleezes™ CC-10, Aquaflex™ SF-40, chitosan derivatives, and polylysine and lysine copolymers.

Other suitable betainic polymers: for example Yukaformere™ (R205, SM) and Diaformer™.

Other suitable copolymers of N-vinylpyrrolidone and vinyl propionate, polysiloxanes, polyethyleneimines and salts thereof, polyvinylamines and salts thereof, cellulose derivatives, polyaspartic acid salts and derivatives.

Also suitable are biopolymers, i.e. polymers and monomers obtained from naturally renewable raw materials and constructed from natural monomer building blocks, e.g. cellulose derivatives, chitin, chitosan, DNA, hyaluronic acid and RNA derivatives, alkyd resins and unsaturated triglycerides.

The cosmetic or dermatological preparations are prepared by the customary principles which are known to the person skilled in the art.

Such formulations are advantageously in the form of emulsions, preferably as water-in-oil (W/O) or oil-in-water (O/W) emulsions. It is, however, also possible according to the invention and in some instances advantageous to choose other types of formulation, for example hydrodispersions, gels, oils, oleogels, multiple emulsions, for example in the form of W/O/W or O/W/O emulsions, anhydrous ointments or ointment bases etc.

The emulsions which can be used according to the invention are prepared by known methods.

As well as the copolymer according to the invention, the emulsions comprise customary constituents, such as fatty alcohols, fatty acid esters and, in particular, fatty acid triglycerides, fatty acids, lanolin and derivatives thereof, natural or synthetic oils or waxes and emulsifiers in the presence of water.

The choice of emulsion type-specific additives and the preparation of suitable emulsions is described, for example, in Schrader, Grundlagen und Rezepturen der Kosmetika [Cosmetic Bases and Formulations], Huthig Buch Verlag, Heidelberg, 2^{nd} edition, 1989, third part, to which reference is expressly made here.

Thus, a skin cream which can be used according to the invention can, for example, be in the form of a W/O emulsion. An emulsion of this type comprises an aqueous phase which is emulsified in an oily or fatty phase using a suitable emulsifier system.

The concentration of the emulsifier system in this type of emulsion is between about 4 and 35% by weight, based on the total weight of the emulsion; the fatty phase constitutes between about 20 and 60% by weight, and the aqueous phases between about 20 and 70% by weight, in each case based on the total weight of the emulsion. The emulsifiers are those which are customarily used in this type of emulsion. They are, for example, chosen from: C₁₂-C₁₈ sorbitan fatty acid esters; esters of hydroxystearic acid and C₁₂-C₃₀ fatty alcohols; mono- and diesters of C₁₂-C₁₈ fatty acids and glycerol or polyglycerol; condensates of ethylene oxide and propylene glycols; oxypropylenated/oxyethylenated C₁₂-C₂₀ fatty alcohols; polycyclic alcohols, such as sterols; aliphatic alcohols having a high molecular weight, such as lanolin; mixtures of oxypropylenated/polyglycerolated alcohols and magnesium isostearate; succinyl esters of polyoxyethylenated or polyoxypropylenated fatty alcohols; and mixtures of magnesium, calcium, lithium, zinc or aluminum lanolate and hydrogenated lanolin or lanolin alcohol.

Suitable fatty components which can be present in the fatty phase of the emulsions include hydrocarbon oils, such as paraffin oil, purcellin oil, perhydrosqualene and solutions of microcrystalline waxes in these oils; animal or vegetable oils, such as sweet almond oil, avocado oil, calophylum oil, lanolin and derivatives thereof, castor oil, sesame oil, olive oil, jojoba oil, Karite oil, Hoplostethus oil, mineral oils whose distillation start point under atmospheric pressure is at about 250° C. and whose distillation end point is at 410° C., such as, for example, vaseline oil; esters of saturated or unsaturated fatty acids, such as alkyl myristates, e.g. isopropyl, butyl or cetyl myristate, hexadecyl stearate, ethyl or isopropyl palmitate, octanoic or decanoic triglycerides and cetyl ricinoleate.

The fatty phase can also comprise silicone oils which are soluble in other oils, such as dimethylpolysiloxane, methylphenylpolysiloxane and the silicone glycol copolymer, fatty acids and fatty alcohols.

In order to favor the retention of oils, it is also possible to use waxes, such as, for example, carnauba wax, candellila wax, beeswax, microcrystalline wax, ozokerite wax and Ca, Mg and Al oleates, myristates, linoleates and stearates.

These water-in-oil emulsions are generally prepared by adding the fatty phase and the emulsifier to the batch container. These are then heated at a temperature of from 70 to 75° C., then the oil-soluble ingredients are added and, with stirring, water is added which has been heated beforehand to the same temperature and in which the water-soluble ingredients have been dissolved beforehand; the mixture is stirred until an emulsion of the desired fineness is obtained, which is then left to cool to room temperature, if necessary with occasional stirring.

A care emulsion according to the invention can also be in the form of an O/W emulsion. An emulsion of this type usually comprises an oil phase, emulsifiers which stabilize the oil phase in the water phase, and an aqueous phase, which is usually in thickened form.

The aqueous phase of the O/W emulsion of the preparations according to the invention optionally comprises

alcohols, diols or polyols and ethers thereof, preferably ethanol, isopropanol, propylene glycol, glycerol, ethylene glycol monoethyl ether;

customary thickeners or gel formers, such as, for example, crosslinked polyacrylic acids and derivatives thereof, polysaccharides, such as xanthan gum or alginates, carboxymethylcellulose or hydroxycarboxymethylcellulose, fatty alcohols, polyvinyl alcohol and polyvinylpyrrolidone.

The oil phase comprises oil components which are customary in cosmetics, such as, for example: P1 esters of saturated and/or unsaturated, branched and/or unbranched C₃-C₃₀-alkanecarboxylic acids and saturated and/or unsaturated, branched and/or unbranched C₃-C₃₀-alcohols, of aromatic carboxylic acids and saturated and/or unsaturated, branched and/or unbranched C₃-C₃₀-alcohols, for example, isopropylmyristate, isopropyl stearate, hexyldecyl stearate, oleyl oleate; and also synthetic, semisynthetic and natural mixtures of such esters, such as jojoba oil;

branched and/or unbranched hydrocarbons and hydrocarbon waxes;

silicone oils, such as cyclomethicone, dimethylpolysiloxane, diethylpolysiloxane, octamethylcyclotetrasiloxane and mixtures thereof;

dialkyl ethers;

mineral oils and mineral waxes;

triglycerides of saturated and/or unsaturated, branched and/or unbranched C₈-C₂₄-alkanecarboxylic acids; they can be chosen from synthetic, semisynthetic or natural oils, such as olive oil, palm oil, almond oil or mixtures.

Suitable emulsifiers are, preferably, O/W emulsifiers, such as polyglycerol esters, sorbitan esters or partially esterified glycerides.

The preparation can be carried out by melting the oil phase at about 80° C.; the water-soluble constituents are dissolved in hot water, then slowly added with stirring to the oil phase; the mixture is then homogenized and stirred until cold.

The method according to the invention is also suitable for use in washing and shower gel formulations, and also in bath preparations.

In addition to the polymers listed, such formulations usually comprise anionic surfactants as base surfactants, and amphoteric and nonionic surfactants as cosurfactants, and also lipids, perfume oils, dyes, organic acids, preservatives and antioxidants, and thickeners/gel formers, skin conditioning agents and moisturizers.

In the wash, shower and bath preparations it is possible to use all anionic, neutral, amphoteric or cationic surfactants which are customarily used in body-cleansing compositions.

The formulations comprise from 2 to 50% by weight of surfactants, preferably from 5 to 40% by weight, particularly preferably from 8 to 30% by weight.

Suitable anionic surfactants are, for example, alkyl sulfates, alkylether sulfates, alkylsulfonates, alkylarylsulfonates, alkyl succinates, alkyl sulfosuccinates, N-alkoylsarcosinates, acyl taurates, acyl isethionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

Suitable examples are sodium lauryl sulfate, ammonium lauryl sulfate, sodium lauryl ether sulfate, ammonium lauryl ether sulfate, sodium lauryl sarcosinate, sodium oleyl succinate, ammonium lauryl sulfosuccinate, sodium dodecylbenzene sulfonate, triethanolamine dodecylbenzenesulfonate.

Suitable amphoteric surfactants are, for example, alkylbetaines, alkylamidopropylbetaines, alkylsulfobetaines, alkyl glycinates, alkyl carboxyglycinates, alkyl amphoacetates or alkyl amphopropionates, alkyl amphodiacetates or alkyl amphodipropionates.

For example, cocodimethylsulfopropylbetaine, laurylbetaine, cocamidopropylbetaine or sodium cocamphopropionate can be used.

Suitable nonionic surfactants are, for example, the reaction products of aliphatic alcohols or alkylphenols having 6 to 20 carbon atoms in the alkyl chain which can be linear or branched, with ethylene oxide and/or propylene oxide. The amount of alkylene oxide is about 6 to 60 moles per mole of alcohol. Also suitable are alkylamine oxides, mono- and dialkylalkanolamides, fatty acid esters of polyethylene glycols, ethoxylated fatty acid amides, alkyl polyglycosides or sorbitan ether esters.

In addition, the wash, shower and bath preparations can comprise customary cationic surfactants, such as, for example, quaternary ammonium compounds, for example cetyltrimethylammonium chloride.

In addition, it is also possible to use further customary cationic polymers, such as, for example, copolymers of acrylamide and dimethyldiallylammonium chloride (polyquaternium-7), cationic cellulose derivatives (polyquaternium-4,-10), guar hydroxypropyltrimethylammonium chloride (INCI: Hydroxypropyl Guar Hydroxypropyltrimonium Chloride), copolymers of N-vinylpyrrolidone and quaternized N-vinylimidazole (polyquaternium-16, -44, -46), copolymers of N-vinylpyrrolidone/dimethylaminoethyl methacrylate, quaternized with diethyl sulfate (polyquaternium-11) and others.

Furthermore, the wash and shower gel formulations and bath preparations can comprise thickeners, such as, for example, sodium chloride, PEG-55, propylene glycol oleate, PEG-120 methyl glucose dioleate and others, and also preservatives, further active ingredients and auxiliaries and water.

In the case of the use according to the invention in hair cosmetics, specifically in the case of use as setting agents, it is advantageous to use formulations from which, following application, a glass transition temperature of greater than 20° C. results.

### EXAMPLES

The formulations listed below are

1. applied to tresses of hair in the customary manner and irradiated with a NIR radiation source (NIR unit model MPP-120-10 from Industrie Servis, D-83052 Bruckmühl) for 10 min from a distance of 25 cm. The intensity of the NIR radiation source is modulated such that no overheating takes place on the substrate surface. Following irradiation, all the tresses of hair display high shape stability. The tresses of hair treated in accordance with the invention are more shape-stable after spraying with water, in particular, than those treated with conventional hair-setting agents without NIR radiation. In the case of irradiation with NIR radiation, the tresses of hair warm up less than in the case of irradiation with a conventional IR radiation source.

2. applied to the fingernails of subjects in a manner customary for nail varnishes, and irradiated as above. Films form which have an improved scratch resistance compared with nail varnish dried conventionally.

3. applied to collagen film, which serves as a model for human skin, and irradiated as above. The collagen film has been preswollen for 24 h in water at 23° C. The gravimetric comparison of untreated and treated film shows that the treated film releases less moisture than the untreated film.

### Example 1

Hair-setting composition

1.50 g of polyurethane acrylate (Laromer LR 8987, BASF, Ludwigshafen/Rh.)

1.50 g of vinylpyrrolidone/vinyl acetate copolymer (Luviskol VA 64, BASF, Ludwigshafen/Rh.)

0.20 g of 1,2-propylene glycol

0.15 g of perfume

0.03 g of cetyltrimethylammonium chloride

0.008 g of cumyl peroxyneodecanoate (as aqueous emulsion, Trigonox 99-W40, Akzo Nobel Chemicals, Amersfoort, NL)

20.21 g of water

76.41 g of ethanol

### Example 2

Coloring hair-setting composition

0.88 g of oligoether acrylate (LR 8863, BASF, Ludwigshafen/Rh.)

2.63 g of vinylpyrrolidone/vinyl acetate copolymer (Luviskol VA 73, BASF, Ludwigshafen/Rh.)

0.20 g of 1,2-propylene glycol

0.15 g of perfume

0.05 g of cetyltrimethylammonium chloride

0.005 g of 2,2′-azobis(2,4,4-trimethylpentane), (VR-110 from Wako, Osaka, JP)

0.05 g of Basic Brown 17 (C. I. 12 251)

0.01 g of Basic Blue 7 (C. I. 42 595)

0.002 g of Basic Violett 14 (C. I. 42 510)

59.89 g of water

46.28 g of ethanol

### Example 3

80 % VOC pump spray

1.50 g of inorganic-organic hybrid prepolysiloxane from mercaptopropyltriethoxysilane, vinyltriethoxysilane and aqueous hydrochloric acid (1 N) (as in DE 19822722 A1, system 11.)

1.50 g of inorganic-organic hybrid prepolysiloxane from 3-glycidoxypropyltrimethoxysilane, tetramethoxysilane, tributoxyaluminum, tetrapropoxyzirconium and triethanolamine (as in DE 19822722 A1, system 3.)

5.00 g of vinyl acetate/crotonic acid/polyethylene oxide copolymer

0.30 g of perfume

11.70 g of water

80.00 g of ethanol

### Example 4

Hair-setting composition with UV protection

1.00 g of polyester acrylate (Laromer LR 8895, BASF, Ludwigshafen/Rh.)

2.00 g of polyvinylpyrrolidone (Luviskol, BASF, Ludwigshafen/Rh.)

0.20 g of perfume

0.15 g of glycerol (85 percent)

0.10 g of 2-hydroxy-4-methoxybenzophenone

0.01 g of 2,2′-azobis[2-methyl-N-(2-hydroxyethyl)propionamide] (VA-086 from Wako, Osaka, JP)

61.30 g of water

35.25 g of ethanol

### Example 5

Hair-setting composition

1.12 g of glycerol aldehyde resorcinol precondensate as in U.S. Pat. No. 4,278,659, formulation 1

1.00 g of refined linseed oil

2.12 g of polyvinylpyrrolidone (Luviskol, BASF, Ludwigshafen/Rh.)

0.40 g of hydrogenated castor oil, ethoxylated with 40 mol of ethylene oxide

0.10 g of tert-butyl hydroperoxide (in the form of a 70% aqueous solution, Trigonox A-W70, Akzo Nobel Chemicals, Amersfoort, NL)

0.03 g of manganese(II) acetate

0.20 g of perfume

95.16 g of water

### Example 6

Setting foam with strong hold

1.00 g of trimethylolpropane ethoxylate-20 triacrylate (Sartomer SR 415, Sartomer, Exton, Pa.)

1.00 g of bisphenol A ethoxylate-30 dimethacrylate (Sartomer SR 9036, Sartomer, Exton, Pa.) 2.00 g of vinylpyrrolidone/methylaminoethyl methacrylate copolymer (Copolymer 845, ISP, Wayne, NJ) 0.45 g of glyceryl laurate 0.15 g of perfume 0.16 g of cetyltrimethylammonium chloride 0.02 g of 2,2′-azobis(2-amidinopropane) dihydrochloride (V-50 from Wako, Osaka, JP) 5.00 g of propane/butane (5.0 bar) 14.95 g of ethanol 75.29 g of water

### Example 7

Setting foam

2.00 g of aromatic polyurethane acrylate (Laromer LR 8949, BASF, Ludwigshafen/Rh.)

1.00 g of aliphatic polyurethane acrylate (Laromer LR 8983, BASF, Ludwigshafen/Rh.)

1.00 g of vinylpyrrolidone/methylaminoethyl methacrylate copolymer (Copolymer 958, ISP, Wayne, N.J.)

0.20 g of 1,2-propylene glycol

0.17 g of perfume

0.10 g of cetyltrimethylammonium chloride

6.00 g of propane/butane (5.0 bar)

0.02 g of 2,2′-azobis(N,N′-dimethyleneisobutyramidine) (VA-061 from Wako, Osaka, JP)

18.66 g of ethanol

70.87 g of water

### Example 8

Setting foam

2.00 g of poly(ethylene glycol) bis(epoxypropyl ether) (Aldrich, Deisenhofen)

2.00 g of chitosan

2.00 g of polyvinylpyrrolidone (Luviskol K 30, BASF, Ludwigshafen/Rh.)

0.20 g of 1,2-propylene glycol

0.17 g of perfume

0.10 g of cetyltrimethylammonium chloride

6.00 g of propane/butane (5.0 bar)

0.03 g of tert-butyl peroxybenzoate (in the form of the 50% strength solution in isododecane, Trigonox F-C50, Akzo Nobel Chemicals, Amersfoort, NL)

69.04 g of ethanol

19 19.49 g of water

### Example 9

Care setting foam

1.12 g of polyurethane acrylate dispersion as in DE 19816527 A1, formulation I.1

1.12 g of polyurethane acrylate dispersion as in DE 19816527 A1, formulation II.1

3.40 g of vinylcaprolactam/vinylpyrrolidone/dimethylaminoethyl methacrylate terpolymer (Copolymer VC 713, ISP, Wayne, N.J.)

0.60 g of formic acid

0.60 g of hydrogenated castor oil, ethoxylated with 40 mol of ethylene oxide

0.22 g of decyl polyglucoside

0.09 g of cetyltrimethylammonium chloride

0.20 g of perfume

6.00 g of propane/butane (5.0 bar)

87.77 g of water

### Example 10

Styling hair spray

1.14 g of amine-modified polyurethane acrylate(Laromer 8869, BASF, Ludwigshafen/Rh.)

1.00 g of oligoether acrylate (Laromer 8967, BASF, Ludwigshafen/Rh.)

1.00 g of polyester acrylate (PE 55 F, BASF, Ludwigshafen/Rh.)

1.50 g of octylacrylamide/butylaminoethyl methacrylate/methacrylate copolymer (Amphomer 28-4910, National Starch, Bridgewater, N.J.)

0.15 g of perfume

0.04 g of 2,5-bis(tert-butylperoxy)-2,5-dimethyl-3-hexyne (Trigonox 145-E85, Akzo Nobel Chemicals, Amersfoort, NL)

0.02 g of dimethyl 2,2′-azobis(isobutyrate) (V-601 from Wako, Osaka, JP)

10.67 g of butane (1.5 bar)

33.33 g of propane/butane

51.21 g of ethanol

### Example 11

Pump spray

2.79 g of polyester acrylate (PE 55 W, BASF, Ludwigshafen/Rh.)

0.30 g of perfume

0.10 g of dimethylsiloxane/ethylene glycol copolymer (Belsil DMC 6032, Wacker, Burghausen)

0.02 g of tert-butyl hydroperoxide (in the form of the 70% aqueous solution, Trigonox A-W70, Akzo Nobel Chemicals, Amersfoort, NL)

11.53 g of water

85.28 g of ethanol

### Example 12

80% VOC hair spray

2.45 g of aromatic polyurethane acrylate (Laromer LR 8983, BASF, Ludwigshafen/Rh.)

4.00 g of vinyl acetate/crotonic acid/vinyl propionate copolymer (Luviset CAP, BASF, Ludwigshafen/Rh.)

0.20 g of cyclopenta(dimethylsiloxane)

0.15 g of perfume

0.10 g of bis(3,5,5-trimethylhexanoyl) peroxide (in the form of the 50% aqueous emulsion, Trigonox 36 W, Akzo Nobel Chemicals, Amersfoort, NL)

13.20 g of water

40.00 g of ethanol

40.00 g of dimethyl ether

### Example 13

Pump spray

2.79 g of polyester acrylate (PE 55 W, BASF, Ludwigshafen/Rh.)

0.30 g of perfume

0.20 g of dimethylsiloxane/ethylene glycol copolymer (Belsil DMC 6032, Wacker, Burghausen)

0.02 g of NIR initiator (No. 1)

11.53 g of water

85.28 g of ethanol

### Example 14

Pump spray

0.50 g of alkyd resin (Alkydal F 300, Bayer, Leverkusen)

1.00 g of caprolactone acrylate (Sartomer SR 495, Sartomer, Exton, Pa.)

0.80 g of polyethylene glycol diallyl ether (BASF, Ludwigshafen/Rh.)

1.20 g of dipentaerythritol pentaacrylate (Sartomer SR 399, Sartomer, Exton, Pa.)

0.30 g of perfume

0.10 g of dimethylsiloxane/ethylene glycol copolymer (Belsil DMC 6032, Wacker, Burghausen)

0.1 g of tert-butyl hydroperoxide (in the form of the 70% aqueous solution, Trigonox A-W70, Akzo Nobel Chemicals, Amersfoort, NL)

0.05 g of manganese(II) acetate

11.53 g of water

85.28 g of ethanol

### Example 15

Setting hairstyling gel

1.53 g of inorganic-organic hybrid prepolysiloxane from mercaptopropyltriethoxysilane and hydrochloric acid (as in DE 19822722, system 9)

2.50 g of polyvinylpyrrolidone

2.10 g of hydroxypropyl guar

0.80 g of hydrogenated castor oil, oxyethylated with 45 mol of ethylene oxide

0.45 g of sodium benzoate

0.30 g of hydroxyethylcellulose

0.20 g of perfume

0.09 g of sodium formate

0.05 g of mica/titanium oxide/tin oxide powder (Soloron® Silver Sparkle from Merck, Germany)

91.98 g of water

### Example 16

Hair cocktail

Phase A

3.00% by weight of Luvigel EM™ (BASF)

2.00% by weight of Belsil DM 1000™ (Wacker)

3.00% by weight of Belsil CM 1000™ (Wacker)

2.00% by weight of Belsil PDM 200™ (Wacker)

2.00% by weight of Belsil ADM 6057 E™ (Wacker)

0.50% by weight of Belsil DMC 6031™ (Wacker)

1.00% by weight of Macadamia nut oil (e.g. Huile de Macadamio from Wacker)

0.50% by weight of vitamin-E-acetate™ (BASF)

1.00% by weight of Cremophor RH 40™ (BASF)

0.40% by weight of perfume oil

Phase B

4.00% by weight of polymer as in Example 14

0.46% by weight of 2-amino-2-methylpropanol

0.10% by weight of Euxyl K 100™(Schulke & Mayr) ad 100.00 of water, demineralized

### Example 17

Hair repair

6.00% of Luviflex Silk™ (BASF)

0.69% of 2-amino-2-methylpropanol

0.20% of hydrolyzed wheat protein (e.g. Cropesol W™ from Croda, Inc.)

0.50% of D-panthenol USP™ (BASF)

5.00% of 1,2-propylene glycol USP™ (BASF)

10,00% of ethanol

77.61% of water, demineralized.

### Example 18

Skin cream

A water/oil cream emulsion (skin cream A) according to the invention was firstly prepared, in accordance with the following recipe:

| Additive | | % by wt. |
|---|---|---|
| Cremophor A 6 | ceteareth-6 and stearyl alcohol | 2.0 |
| Cremophor A 25 | ceteareth-25 | 2.0 |
| Lanette O | cetearyl alcohol | 2.0 |
| Imwitor 960 K | glyceryl stearate SE | 3.0 |
| Paraffin oil | | 5.0 |
| Jojoba oil | | 4.0 |
| Luvitol EHO | cetearyl octanoate | 3.0 |
| ABIL 350 | dimethicone | 1.0 |
| Amerchol L 101 | mineral oil and lanolin alcohol | 3.0 |
| Veegum Ultra | magnesium aluminum silicate | 0.5 |
| 1,2-Propylene glycol | propylene glycol | 5.0 |
| Abiol | imidazolindinylurea | 0.3 |
| Phenoxyethanol | | 0.5 |
| D-Panthenol USP | | 1.0 |
| Polymer (Luviskol VA 63, | | 0.5 |
| BASF) | | |
| Water | | ad 100 |

### Example 19

Shower gel

A shower gel formulation (shower gel A) according to the invention was firstly prepared in accordance with the following recipe:

| Additive | | % by wt. |
|---|---|---|
| Texapon NSO | sodium laurethsulfate | 40.0 |
| Tego Betaine L7 | cocamidopropylbetaine | 5.0 |
| Plantacare 2000 | decyl glucoside | 5.0 |
| Perfume | | 0.2 |
| Polymer as in Example 15 | | 0.2 |
| Euxyl K 100 | benzyl alcohol, methylchloro- | 0.1 |
| | isothiazolinone, methylisothia- | |
| | zolinone | |
| D-Panthenol USP | | 0.5 |
| Citric acid (pH 6-7) | | q.s. |
| NaCl | | 2.0 |
| Water | | ad 100 |

### Example 20

Humectant formulation

Formulation A

| Additive | | | % by wt. |
|---|---|---|---|
| a) | Cremophor A6 | ceteareth-6 and stearyl | 2.0 |
| | | alcohol | |
| | Cremophor A25 | ceteareth-25 | 2.0 |
| | Paraffin oil (viscous) | | 10.0 |
| | Lanette O | cetearyl alcohol | 2.0 |
| | Stearic acid | | 3.0 |
| | Nip-Nip | methylparaben/propyl | 0.5 |
| | | paraben 70:30 | |
| | Abiol | Imidazoldinylurea | 0.5 |
| b) | Polymer (Hypermer B 246, | | 3.0 |
| | ICI) | | |
| | Water | | ad 100 |

The two phases were heated to 80° C., phase a) was stirred into b), homogenized and stirred until cold, and then adjusted to pH 6 with 10% strength aqueous NaOH solution.

### Example 21

O/W cream for retaining moisture

| Additive | % by wt. |
|---|---|
| Glycerol monostearate | 2.0 |
| Cetyl alcohol | 3.0 |
| Paraffin oil, subliquidum | 15.0 |
| Vaseline | 3.0 |
| Caprylic/capric triglyceride | 4.0 |
| Octyldodecanol | 2.0 |
| Hydrogenated coconut fat | 2.0 |
| Cetyl phosphate | 0.4 |
| Polymer (Example 18) | 3.0 |
| Glycerol | 3.0 |
| Sodium hydroxide | q.s. |
| Perfume oil | q.s. |
| Preservative | q.s. |
| Water | ad 100 |

### Example 22

O/W lotion

| Additive | % by wt. |
|---|---|
| Stearic acid | 1.5 |
| Sorbitan monostearate | 1.0 |
| Sorbitan monooleate | 1.0 |
| Paraffin oil, subliquidum | 7.0 |
| Cetyl alcohol | 1.0 |
| Polydimethylsiloxane | 1.5 |
| Glycerol | 3.0 |
| Polymer (Example 20) | 0.5 |
| Perfume oil | q.s. |
| Preservative | q.s. |
| Water | ad 100 |

### Example 23

W/O cream

| Additive | % by wt. |
|---|---|
| PEG-7 hydrogenated castor oil | 4.0 |
| Woolwax alcohol | 1.5 |
| Beeswax | 3.0 |
| Triglyceride, liquid | 5.0 |
| Vaseline | 9.0 |
| Ozokerite | 4.0 |
| Paraffin oil, subliquidum | 4.0 |
| Glycerol | 2.0 |
| Polymer (Example 20) | 2.0 |
| Magnesium sulfate*7H₂O | 0.7 |
| Perfume oil | q.s. |
| Preservative | q.s. |
| Water | ad 100 |

### Example 24

Hydrogel for skincare

| Additive | % by wt. |
|---|---|
| Polymer (Preparation Example 19) | 3.0 |
| Sorbitol | 2.0 |
| Glycerol | 3.0 |
| Polyethylene glycol 400 | 5.0 |
| Ethanol | 1.0 |
| Perfume oil | q.s. |
| Preservative | q.s. |
| Water | ad 100 |

### Example 25

Hydrodispersion gel

| Additive | % by wt. |
|---|---|
| Polymer (Preparation Example 19) | 3.0 |
| Sorbitol | 2.0 |
| Glycerol | 3.0 |
| Polyethylene glycol 400 | 5.0 |
| Triglyceride, liquid | 2.0 |
| Ethanol | 1.0 |
| Perfume oil | q.s. |
| Preservative | q.s. |
| Water | ad 100 |

### Example 26

Liquid soap

| Additive | % by wt. |
|---|---|
| Coconut fatty acid, potassium salt | 15 |
| Potassium oleate | 3 |
| Glycerol | 5 |
| Polymer (Preparation Example 14) | 2 |
| Glycerol stearate | 1 |
| Ethylene glycol distearate | 2 |
| Specific additives, complexing agents, fragrances | q.s. |
| Water | ad 100 |

### Example 27

Body care cream

| Additive | | % by wt. |
|---|---|---|
| Cremophor A6 | ceteareth-6 and stearyl alcohol | 2.0% |
| Cremophor A 25 | ceteareth-25 | 2.0% |
| Grape (Vitis vinifera) | | 6.0% |
| seed oil | | |
| Glyceryl stearate SE | | 3.0% |
| Cetearyl alcohol | | 2.0% |
| Dimethicone | | 0.5% |
| Luvitol EHO | cetearyl octanoate | 8.0% |
| Oxynex 2004 | propylene glycol, BHT, ascorbyl | 0.1% |
| | palmitate, glyceryl stearate, | |
| | citric acid | |
| Preservative | | q.s. |
| 1,2-Propylene glycol USP | | 3.0% |
| Glycerol | | 2.0% |
| EDTA BD | | 0.1% |
| D-Panthenol USP | | 1.0% |
| Water | | ad 100 |
| Polymer (Example 20) | | 1.5% |
| Tocopheryl acetate | | 0.5% |

The formulation had a pH of 6.8. The viscosity (Brookfield RVT, 23° C.) was 32000 mPas.

### Example 28

Mascara (Wimperntusche)

| | | |
|---|---|---|
| Phase A | 1.50% | of Cremophor A6 ™ (BASF) |
| | 1.50% | of Cremophor A25 ™ (BASF) |
| | 2.00% | of stearic acid (e.g. Emersol 120 ™ from |
| | | Henkel) |
| | 3.00% | of Imwitor 960 K ™ (Hüls AG) |
| | 3.00% | of Softisan 100 ™ (Hüls AG) |
| | 1.50% | of Luvigel EM ™ (BASF) |
| | 10.00% | of Dow Corning 345 ™ (Dow Corning) |
| Phase B | 4.00% | of Luviflex Silk ™ (BASF) |
| | 0.46% | of 2-amino-2-methylpropanol |
| | 0.30% | of Germal 115 ™ (Sutton) |
| | 72.24% | of water, demineralized |
| Phase C | 0.50% | of phenoxyethanol (e.g. Phenoxetol ™ from |
| | | Nipa-Hardwicke) |

### Example 29

Make-up

| | | | | |
|---|---|---|---|---|
| A | 2.0 | of Cremophor A 6 | (1) | Ceteareth-6, Stearyl |
| | | | | Alcohol |
| | 2.0 | of Cremophor A 25 | (1) | Ceteareth-25 |
| | 4.0 | of cetylstearyl alcohol | | Cetearyl Alcohol |
| | 6.0 | of Luvitol EHO | (1) | Cetearyl Octanoate |
| B | 4.0 | of 1,2-propylene glycol USP | (1) | Propylene Glycol |
| | 2.0 | of D-panthenol 50 P | (1) | Panthenol, Propylene |
| | | | | Glycol |
| | 0.5 | of active ingredient | | |
| | | concentration of polymer | | |
| | q.s. | of neutralizing agent (if | | |
| | | necessary) | | |
| | q.s. | of preservative | | |
| | ad 100 | with water, dem. | | |
| C | q.s. | of vitamins | | |
| | q.s | of perfume oil | | |

Preparation

Heat phases A and B separately to about 80° C., stir phase A into phase B and homogenize. Cool to about 40° C. with stirring, incorporate phase C and homogenize again.

### Example 30

Sunscreen emulsion

| | | | | |
|---|---|---|---|---|
| | 1.00 | of Cremophor A 6 | (1) | Ceteareth-6, Stearyl |
| | | | | Alcohol |
| | 2.00 | of Cremophor A 25 | (1) | Ceteareth-25 |
| | 3.00 | of glycerol monostearate | (44) | Glyceryl Stearate |
| | 2.00 | of cetylstearyl alcohol | (27) | Cetearyl Alcohol |
| | 2.00 | of Luvitol EHO | (1) | Cetearyl Octanoate |
| | 1.00 | of Uvinul T 150 | (1) | Octyl Triazone |
| | 5.00 | of Uvinul MC 80 | (1) | Octyl Methoxycinna- |
| | | | | mate |
| | 3.00 | of Uvinul MBC 95 | (1) | 4-Methylbenzylidene |
| | | | | Camphor |
| | 5.00 | of Z-Cote | (1) | Zinc Oxide |
| | 7.00 | of isopropyl myristate | (27) | Isopropyl Myristate |
| B | 0.20 | EDTA BD | (1) | EDTA |
| | 0.50 | D-Panthenol USP | (1) | Panthenol |
| | 5.00 | 1,2-Propylene glycol USP | (1) | Propylene Glycol |
| | 7.50 | Luviquat Care | (1) | Polyquaternium-44 |
| | q.s. | Preservative | | |
| | 54.50 | Water, dem. | | Aqua dem. |
| C | 0.30 | Keltrol T | (66) | Xanthan Gum |
| D | 1.00 | Vitamin E acetate | (1) | Tocopheryl Acetate |
| | q.s. | Perfume oil | | |

Suppliers
(1) BASF Aktiengesellschaft
(27) Cognis Deutschland GmbH
(44) Th. Goldschmidt AG
(66) Kelco International GmbH

We claim:

1. A method of treating a cosmetic composition, which comprises treating the cosmetic composition with NIR radiation before, during or after application.

2. A method as claimed in claim 1, wherein the emission maximum of the NIR radiation is in the wavelength range from 600 to 1500 nm.

3. A method as claimed in claim 1 or 2, wherein the cosmetic composition is used for the treatment of skin, hair and/or nails.

4. A method as claimed in any of claims 1 to 3, wherein the cosmetic composition has film-forming or permeation-inhibiting properties.

5. A method as claimed in any of claims 1 to 4, wherein the cosmetic composition comprises at least one polymer.

6. A method as claimed in any of claims 1 to 5, wherein, as a result of the action of NIR radiation, at least one polymer is formed and/or the molecular weight of at least one polymer is increased and/or the properties of the polymer are changed.

7. A method as claimed in any of claims 1 to 6, wherein the polymer is a poly(meth)acrylate, polyamide, polyester, polyether, polyurethane, poly-N-vinyllactam, polyolefin, polyvinyl ester, polysiloxane or a copolymer comprising repeat units of the abovementioned polymers.

8. A method as claimed in any of claims 1 to 7, wherein the polymer has at least one repeat unit which comes from compounds of the group methyl methacrylate, tert-butyl acrylate, 2-butyl methacrylate, styrene, N-tert-butylacrylamide, vinyl acetate, vinyl propionate, vinylcaprolactam, vinylpyrrolidone, vinylimidazole and N-methylvinylimidazolinium salt, ethyl acrylate, methyl acrylate, hydroxyethyl acrylate, n-butyl acrylate, lauryl (meth)acrylate, stearyl (meth)acrylate, vinyl neodecanoate, acrylic acid, methacrylic acid, crotonic acid.

9. A method as claimed in any of claims 1 to 8, wherein the polymer is a polyelectrolyte.

10. A method as claimed in any of claims 1 to 9, wherein the polymer is formed by polymerization, polyaddition or polycondensation or a combination of these processes.

11. A method as claimed in any of claims 1 to 10, wherein the intensity of the radiation can be matched to the purpose of the treatment.

12. A method as claimed in any of claims 1 to 11, wherein the radiation is produced by a plurality of radiation sources, where the type and intensity of the radiation sources may be identical or different.

13. A method as claimed in any of claims 1 to 12, wherein the radiation sources are positioned equidistant to the head.

14. A method as claimed in any of claims 1 to 13, wherein the radiation sources are positioned equidistant to one another.

15. A cosmetic composition obtainable by irradiation with NIR radiation.

16. A cosmetic composition as claimed in claim 15, wherein the cosmetic composition comprises at least one ethylenically unsaturated compound, which may be either of low molecular weight or of high molecular weight.

17. A cosmetic composition as claimed in claim 15 or 16, wherein the emission maximum of the NIR radiation is in the wavelength range from 600 to 1500 nm.

18. A cosmetic composition as claimed in any of claims 15 to 17, wherein at least one polymer is formed under the action of NIR radiation.

19. A cosmetic composition as claimed in any of claims 15 to 18, wherein the cosmetic composition comprises at least one initiator, dye or catalyst which permits, accelerates or intensifies the action of the radiation.

20. A cosmetic composition as claimed in any of claims 15 to 19, wherein the cosmetic composition comprises at least one initiator for a free-radical polymerization.

21. A cosmetic composition as claimed in any of claims 15 to 20, wherein the cosmetic composition comprises at least one inorganic compound having a 0 -0 bond, and optionally reducing agents.

22. A cosmetic composition as claimed in any of claims 15 to 21, wherein the cosmetic composition comprises at least one low molecular weight or high molecular weight compound which has at least one, and preferably two to eight, functionalities from the group consisting of —OH, —NHR, —COOR, —C(H)═O, epoxide, —NCO, where R is hydrogen or any organic radical.

23. A cosmetic composition as claimed in any of claims 15 to 22, wherein the cosmetic composition comprises compounds chosen from the group consisting of (meth)acrylate-functional (meth)acrylic copolymers, polyether (meth)acrylates, polyester (meth)acrylates, unsaturated polyesters, epoxy (meth)acrylates, urethane (meth)acrylates, amino(meth)acrylates, melamine (meth)acrylates, silicone (meth)acrylates as well as customary additives.

24. A cosmetic composition as claimed in any of claims 15 to 23, comprising, as well as customary additives, a further anionic, cationic. neutral or betainic polymer, a biopolymer or a prepolymer.

25. The use of a cosmetic composition as claimed in any of claims 15 to 24 for the treatment of skin, hair or nails.

26. The use of the cosmetic composition as claimed in claim 25 for the treatment of hair.

27. The use of the cosmetic composition as claimed in claim 25 or 26, wherein the cosmetic composition is chosen from the group consisting of permanent wave preparations, hair treatments, hair lotions, hair rinses, hair emulsions, split-end fluids, neutralizers for permanent waves, hot-oil treatment preparations, conditioners, setting lotions, shampoos, hair colorants, hair sprays, setting foams, hair mousse, hair gel, agents for treating dandruff and hair loss, and hair restorers.

28. The use of the cosmetic composition as claimed in claim 25 for the treatment of nails.

29. The use of the cosmetic composition as claimed in claim 28, wherein the composition for the treatment of nails is chosen from the group consisting of nail care compositions and nail varnishes.

30. The use of the cosmetic composition as claimed in claim 25, wherein the composition is chosen from cosmetic compositions for the treatment of skin and wound treatment.

31. The use of the cosmetic composition as claimed in claim 30, wherein the composition for the treatment of skin is chosen from the group consisting of W/O or O/W skin creams, day and night creams, eye creams, face creams, antiwrinkle creams, moisturizing creams, bleaching creams, vitamin creams, skin cleansers, babycare compositions, and decorative cosmetics.

32. The use of the cosmetic composition as claimed in any of claims 25 to 31, wherein, following irradiation with NIR radiation, a glass transition temperature of the composition of greater than 20° C. results.
